(19) United States
(12) Patent Application Publication
(10) Pub. No.: US 2016/0361509 A1
(43) Pub. Date: Dec. 15, 2016

(54) SECURING MECHANISMS FOR ENDOTRACHEAL TUBES

(71) Applicant: Dale Medical Products, Inc., Plainville, MA (US)

(72) Inventors: William Ray Blessing, JR., Manitou Springs, CO (US); Anthony Mullane, Colorado Springs, CO (US); Joshua Brezack, Franklin, MA (US)

(21) Appl. No.: 15/179,449

(22) Filed: Jun. 10, 2016

Related U.S. Application Data
(60) Provisional application No. 62/173,561, filed on Jun. 10, 2015.

(51) Int. Cl. A61M 16/04; A61M 16/08

(52) U.S. Cl. CPC A61M 16/0497; A61M 16/0493; A61M 16/0816; A61M 16/0463; A61M 2205/02

(57) ABSTRACT

Embodiments of the present disclosure provide an endotracheal tube holder and a band-locking mechanism. The endotracheal tube holder has an elongated body portion and a tube-grasping portion. The body portion may have a band-locking mechanism to secure the endotracheal tube holder to a patient. The tube-grasping portion has a tube clamp having a bite-block portion, a tube-securing portion, and at least one channel separating the bite-block portion from the tube-securing portion. The tube securing portion further has a locking assembly and the tube-grasping portion further includes at least one hinge.

### TECHNICAL FIELD

The present disclosure relates to securing mechanisms, which may be used more specifically with endotracheal tubes.

### BACKGROUND

Endotracheal intubation, placing a flexible tube into the trachea to maintain an open airway or to serve as a conduit through which to administer certain drugs, is a medical necessity in a variety of settings including in critical care, intensive care units, or under general anesthesia. One setting that presents unique challenges is in emergency or “field” settings, such as ambulances or battlefields, where increased usability is needed. Endotracheal intubation is commonly accomplished by inserting an endotracheal tube via the mouth or nose to a sufficient depth in the trachea and holding the endotracheal tube in position. Displacement or movement of the endotracheal tube by the medical practitioner or the patient can create problems that reduce or lose the effectiveness of the artificial airway, leading to further medical emergency and even to loss of life. Therefore, securing, fixating, and/or stabilizing the endotracheal tube in the correct position help facilitate effective medical procedures and prevents complications.

A common practice for securing and maintaining the position of the endotracheal tube is to tape the tube to the face of a patient. This method has many disadvantages, including time-consuming operational setup, discomfort for patient, difficulty of removing the tube, and little opportunity for readjustment. Other tube-securing methods include complex mounting plates and applying pressure to the face of a patient and/or using straps to secure the plate to the patient. Oftentimes, the complex configurations of these mounting plates require more than one medical practitioner to set up and secure the plate to the patient. Furthermore, these complex mounting plates obstruct clear entry into the mouth of a patient, preventing other critical medical procedures, such as suction, inspection, oral care, and maintaining cleanliness of the endotracheal tube and the integrity of tissues and surfaces involved. Additionally, in current medical practice, an auxiliary tube with a balloon cuff is typically provided with an endotracheal tube and inflated after intubation to help secure the endotracheal tube and seal the tracheobronchial tree, preventing leakage of respiratory gases and contamination by undesirable materials. However, the auxiliary tube may interfere with the securing and maintenance procedures of an endotracheal tube and add discomfort to the patient. Therefore, a need exists for easily operable, time-efficient, and patient-friendly securing mechanisms for an endotracheal tube that allow effective and reliable securing of an endotracheal tube to a patient.

### SUMMARY

The inventors have recognized that an endotracheal tube holder that prevents the tube from being moved and/or damaged by a patient's bite is desirable. The inventors also have recognized a need for securing methods of the endotracheal tube holder to the patient that do not require two or more hands. The inventors have further recognized the desirability of an endotracheal tube holder that is adaptable to accommodate and secure medical tubes of wide range of sizes, including the Supraglottic tubes. The inventors have also recognized that it may be advantageous for an endotracheal tube holder to have an auxiliary tube holder that prevents the auxiliary tube from interfering with the securing and maintenance procedures of an endotracheal tube, and that does not limit the positioning of the auxiliary tube with respect to the endotracheal tube holder during deployment.

One embodiment consistent with the present disclosure includes an endotracheal tube holder. The endotracheal tube holder includes an elongated body portion and a tube-grasping portion. The tube-grasping portion includes a tube clamp having a bite-block portion, a tube-securing portion, and at least one channel separating the bite-block portion from the tube-securing portion. The tube-securing portion further includes a locking assembly and the tube-grasping portion further includes at least one hinge.

In certain embodiments of the present disclosure, the endotracheal tube holder includes at least one arm connecting the tube-grasping portion to the body portion. The body portion may further include a reinforcing rib extending along a longitudinal axis of the body portion and connected to the arm. In certain embodiments, the reinforcing rib may be further connected to a base member of the locking assembly.

In certain embodiments of the present disclosure, the reinforcing rib may further include a finger rest that provides leverage for closing the locking assembly.

In certain embodiments of the present disclosure, the tube clamp includes an upper contact member and a lower contact member pivotably connected via the at least one hinge. In some embodiments, a first hinge connects the upper contact member and the lower contact member of the tube-securing portion, and a second hinge connects the upper contact member and the lower contact member of the bite-block portion.

In certain embodiments of the present disclosure, the tube-securing portion includes a plurality of teeth on the interior sides of the upper contact member and lower contact member to secure a tube therebetween. The shapes of the upper contact member and the lower contact member may be partial elliptic cylindrical forms.

In certain embodiments of the present disclosure, at least a part of the interior sides of the upper contact member and/or lower contact member may provide a frictional and/or adhesive surface to increase a gripping force to secure a tube therebetween.

In certain embodiments of the present disclosure, the locking assembly includes a base member and a movable member having a plurality of ratcheting teeth, and the base member is connected to the body portion while the movable member is connected to the tube-securing portion. The base member may operatively latch to the movable member via the plurality of ratcheting teeth.

In certain embodiments of the present disclosure, the body portion includes two widened distal portions at opposite ends for lying against the face of a patient. Moreover, in certain embodiments, at least one of the widened distal portions may include an auxiliary tube holder. In some embodiments, both widened distal portions may include the auxiliary tube holder. In certain embodiments, the auxiliary tube holder may be positioned away from the top and/or bottom edges of the body portion. In certain embodiments, the auxiliary tube holder comprises a notch and orients an auxiliary tube along a longitudinal axis of the body portion.

In certain embodiments of the present disclosure, at least one of the distal portions of an endotracheal tube holder includes a band-locking cleat that secures the endotracheal tube holder to the face of a patient. In certain embodiments, the band-locking cleat includes a first opening having a band-receiving region that receives a band and a band-securing region that friction-fits the band, and a second opening that receives the band from the band-securing region of the first opening. The band may be tightened or loosened around the patient by moving (e.g. pulling) the band through the first opening. In certain embodiments, a channel breaks the second opening to an edge of the distal portion so that the band can be moved into or removed out of the second opening via the channel. In certain embodiments, the band may be secured by moving the band from the band-securing region into the second opening via the channel and released by first removing the band away from the second opening via the channel and then moving the band from the band-securing region to the band-receiving region. In certain embodiments, both of the distal portions have the band-locking cleat. In some embodiments, one distal portion has the band-locking cleat and the other distal portion has the band permanently attached, or alternatively attached via some other mechanism.

In certain embodiments of the present disclosure, the body portion further includes more than two pad regions on a patient-facing side of the body portion. In certain embodiments, the pad regions may be discretely or continuously distributed on the body portion.

Another embodiment consistent with the present disclosure is a device including a band-locking cleat for securing a device to an object or a person. The band-locking cleat includes a first opening having a band-receiving region that receives a band and a band-securing region that friction-fits the band and a second opening that receives the band from the band-securing region of the first opening. In certain embodiments, the band can move through the first opening, and a channel breaks the second opening to an edge of the device so that the band can be moved into or removed out of the second opening via the channel. In certain embodiments, the band may be secured by moving the band from the band-securing region into the second opening via the channel and released by first removing the band away from the second opening via the channel and then moving the band from the band-securing region to the band-receiving region of the first opening.

Another embodiment consistent with the present disclosure includes a method for providing a device having at least one band-locking cleat. The band-locking cleat may include a first opening comprising a band-receiving region that receives a band and a band-securing region that friction-fits the band, and a second opening that receives the band from the band-securing region of the first opening. In certain embodiments, the band can move through the first opening and a channel breaks the second opening to an edge of the device so that the band can be moved into or removed out of the second opening via the channel. Further, in certain embodiments, the band is pre-threaded through the first opening and placed within the band-securing region.

### BRIEF DESCRIPTION OF THE DRAWINGS

The accompanying drawings, which are incorporated in and constitute a part of this specification, illustrate exemplary embodiments consistent with the principles of the present disclosure.

FIG. 1A provides a perspective view of an exemplary endotracheal tube holder consistent with the present disclosure and a pre-threaded band.

FIG. 1B provides a perspective view of another embodiment of the exemplary endotracheal tube holder of FIG. 1A.

FIG. 2 provides a front view of the exemplary endotracheal tube holder of FIG. 1A.

FIG. 3 provides a side view of the exemplary endotracheal tube holder of FIG. 1A.

FIG. 4 provides another view of the exemplary endotracheal tube holder of FIG. 1A.

FIG. 5 provides a rear view of the exemplary endotracheal tube holder of FIG. 1A.

FIG. 6 provides a side view of an assembled endotracheal tube holder holding a tube lying against the face of a patient according to certain embodiments of the present disclosure.

FIG. 7 provides a front view of an exemplary endotracheal tube holder securing a regular-sized endotracheal tube.

FIG. 8 provides a front view of an exemplary endotracheal tube holder securing a Supraglottic tube.

### DESCRIPTION OF EXEMPLARY EMBODIMENTS

Reference will now be made in detail to the present exemplary embodiments, examples of which are illustrated in the accompanying drawings. Wherever possible, the same reference numbers will be used throughout the drawings to refer to the same or like parts.

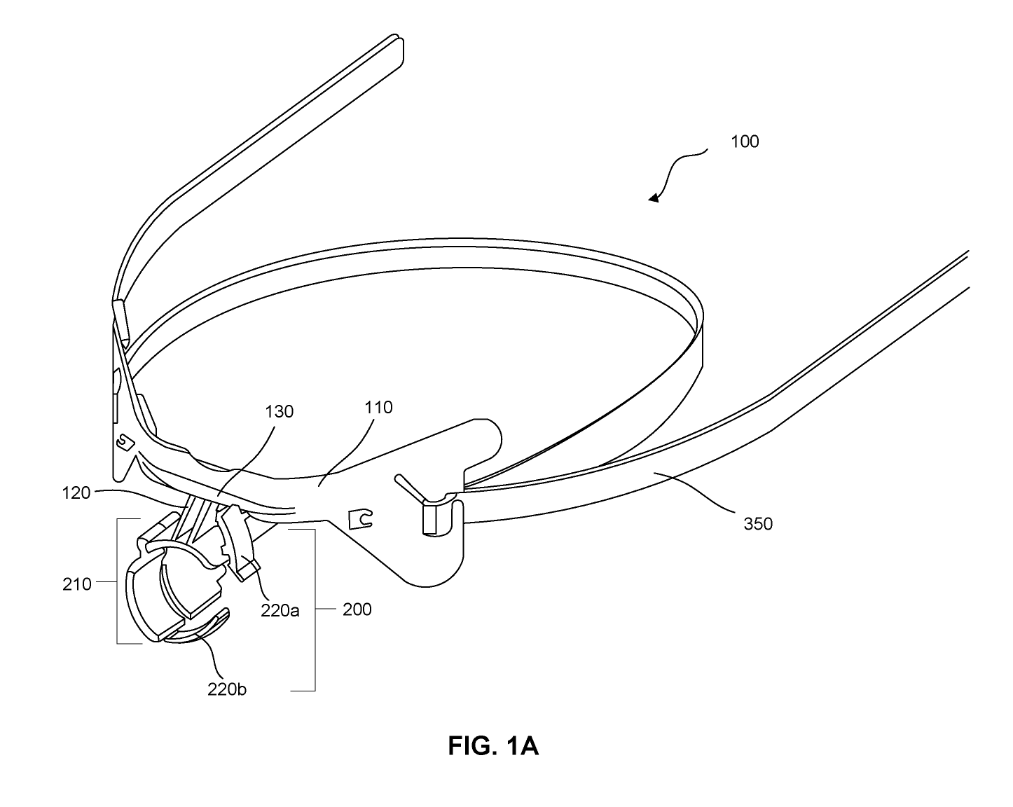

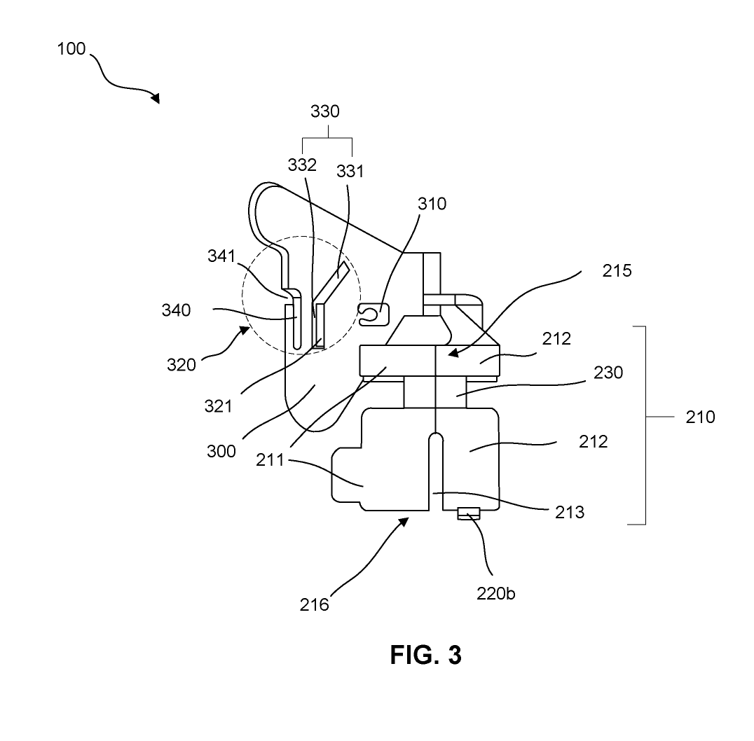

The present disclosure provides endotracheal tube holders that will secure and hold the position of an endotracheal tube to a patient. FIGS. 1A-4 provide various views of an exemplary endotracheal tube holder 100 consistent with the present disclosure. As shown in FIG. 1A, the endotracheal tube holder 100 includes an elongated body portion 110 and a tube-grasping portion 200. The tube-grasping portion 200 further include a tube clamp 210 and a locking assembly 220 (220a and 220b). At least one arm 120 can connect the tube-grasping portion 200 to the body portion 110. The body portion 110 can further include a reinforcing rib 130 extending along a longitudinal axis of the body portion 110 and connected to the at least one arm 120.

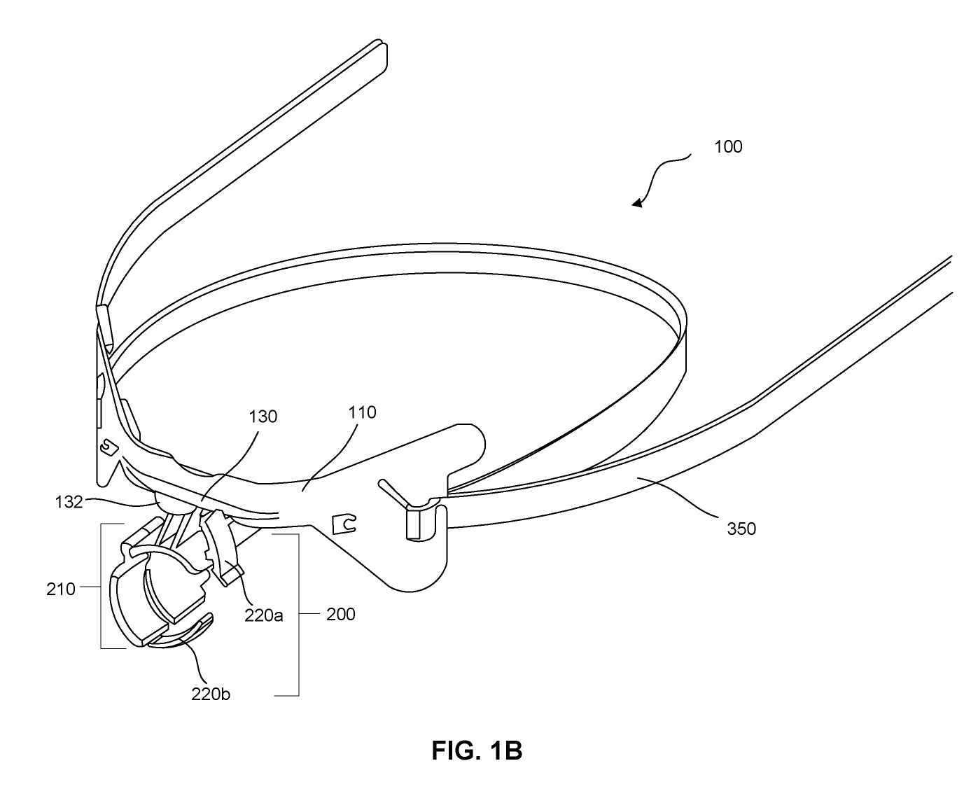

The tube-grasping portion 200 allows secure attachment of an endotracheal tube, thereby preventing unintended movement or dislodgement and can include a variety of different configurations. As shown in FIG. 2, the tube clamp 210 of the tube-grasping portion 200 can include an upper contact member 215 and a lower contact member 216 pivotably connected via at least one hinge 230, which allows the tube clamp 210 to be opened and closed to secure the endotracheal tube in place. The shapes of the upper contact member 215 and the lower contact member 216 may be advantageously designed as partial elliptic cylindrical forms such that the tube clamp 210 can grasp a tube of any size by partially contacting part of the tube's circumference. The endotracheal tube holder 100 thus can adjustably accommodate and secure a variety of tubes having outer diameters ranging from about 7 mm to about 14.2 mm, thus including Supraglottic tubes.

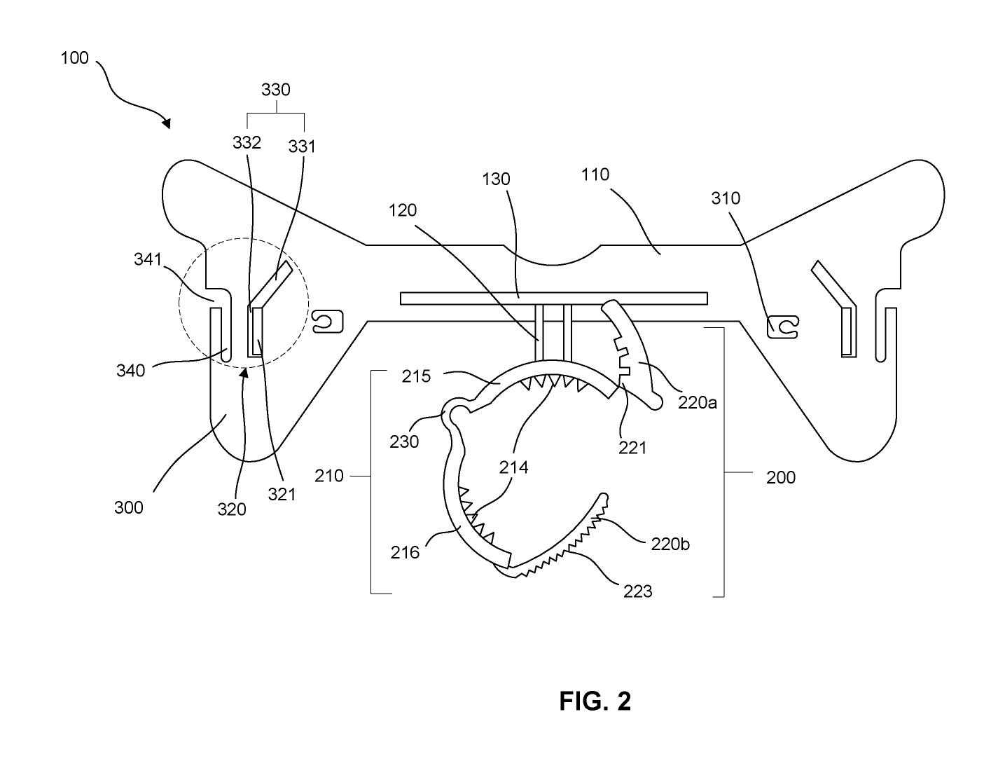

As shown in FIG. 3, the tube clamp 210 has a bite-block portion 211, a tube-securing portion 212, and at least one channel 213 separating the bite-block portion 211 from the tube-securing portion 212. The separation of the bite-block portion 211 from the tube-securing portion 212 by the channel 213 allows the bite-block portion 211 to float freely around a hinge 230 from the tube-securing portion 212 so that the tube-securing portion 212 can maintain the secured position of an endotracheal tube when the endotracheal tube is bitten and/or moved by the patient. In some embodiments, a first hinge may connect the upper contact member 215 and the lower contact member 216 of the tube-securing portion 212, and a second hinge may connect the upper contact member 215 and the lower contact member 216 of the bite-block portion 211. In some embodiments, hinge 230 may be made of a flexible material, and may be an integral part of the bite-block portion 211 and/or the tube-securing portion 212.

As discussed above and shown in FIGS. 1A-3, the tube-grasping portion 200 also includes a locking assembly 220. As shown in FIG. 2, the locking assembly 220 includes a base member 220a preferably connected to the body portion 110 and a movable member 220b preferably connected to the tube-securing portion 212. The base member 220a may include one or more pawls 221, such as bars or levers. The movable member 220b has a plurality of ratcheting teeth 223. The base member 220a can operatively latch to the movable member 220b by engaging the pawls 221 with the plurality of ratcheting teeth 223 to fixate and secure a tube held by the tube clamp 210 at a suitable position. The ratcheting teeth 223 allow closure of the tube clamp 210 with a desired degree of pressure and friction around the endotracheal tube. The reinforcing rib 130 of the body portion 110 is preferably connected to the base member 220a of the locking assembly 220 and can prevent the body portion 110 from flexing, which may cause the movable member 220b of the locking assembly 220 to slide out of the base member 220a of the locking assembly 220.

In some embodiments, as shown in FIG. 1B, the reinforcing rib 130 may include a portion 132 extending from the body portion 110 that provides a finger rest against which a medical practitioner may rest a finger and thus provides leverage for closing the locking assembly 220. For example, to secure an endotracheal tube, a medical practitioner may rest a finger against the surface provided by the extended portion 132 of the reinforcing rib 130, providing support for using other fingers of the same hand to slide the movable member 220b into the base member 220a of the locking assembly 220, while the other hand may be used to hold the endotracheal tube at the same time.

Other tube locking mechanisms may be used in conjunction with the body portion 110 and the tube clamp 210. For example, the tube-grasping portion 200 can include non-rigid closures, such as a flexible band, including VELCRO®. Alternatively, the tube-grasping portion 200 can simply include a flat or curved protrusion to which tape or other connecting materials can be applied. For example, in one embodiment, the tube clamp 210 can receive an endotracheal tube and be closed by using tapes, adhesives, a rigid cap, or other connectors. In some embodiments, the endotracheal tube holder 100 can include a system for adjustably securing the tube-grasping portion 200 to the body portion 110. For example, the elongated body portion 110 may include an elongated opening (not shown) that provides a track along which the tube-grasping portion 200 can be moved to adjust the position of an endotracheal tube along the longitudinal axis of the body portion 110.

As shown in FIG. 2, in some embodiments, the tube clamp 210 may further include a plurality of grabbing teeth 214 on the interior sides of the upper contact member 215 and lower contact member 216, for example, on the tube-securing portion 212 of the tube clamp 210. The grabbing teeth 214 increase friction between contacting regions of the contact members 215 and 216 and the endotracheal tube, thereby reducing or preventing movement or sliding of the endotracheal tube. The placement of the grabbing teeth 214 can conform to the partial elliptic cylindrical shapes of the upper contact member 215 and the lower contact member 216. In various embodiments, the grabbing teeth 214 can be of various sizes and shapes depending on the size of the tube or the specific use.

In some embodiments, at least a part of the interior sides of the upper contact member 215 and/or lower contact member 216 may have a frictional and/or adhesive surface to increase a gripping force for holding an endotracheal tube in place, thereby reducing or preventing movement or sliding of the endotracheal tube by a patient's bite and/or movement. The frictional and/or adhesive surface may be provided in one or more ways. For example, the tube clamp 210 may include a coating or a layer of material (not shown) on the interior sides of the upper contact member 215 and/or lower contact member 216. The coating or layer of material may be made of at least one rubbery, foamy, gummy, and/or adhesive material that have a predetermined frictional and/or adhesive surface texture, thereby increasing the force for gripping an endotracheal tube by the tube clamp 210. Additionally or alternatively, the material of the tube clamp 210 may be injection molded with the rubbery, foamy, gummy, and/or adhesive material such that at least a part of the interior sides of the upper contact member 215 and/or lower contact member 216 may have a frictional and/or adhesive surface to reduce potential movement of the endotracheal tube. The frictional and/or adhesive surface can be provided at either or both of the interior sides of the tube-securing portion 212 and the bite-block portion 211 of the tube clamp 210. In some embodiments, the rubbery, foamy, gummy, and/or adhesive material may be selected from silicon, latex, other suitable polymeric materials, or a combination thereof.

The body portion 110 and the tube-grasping portion 200 can be formed from a variety of different materials, such as FDA-approved high density medical grade polyethylene. In some embodiments, the body portion 110 is formed from a rigid material selected to maintain its curved shape during use. The rigid material may have a curvature selected to conform to the shape of a typical patient's face to allow comfortable placement of the endotracheal tube holder. In some embodiments the body portion 110 is formed from a flexible material to conform to the shape of a patient's face to allow comfortable placement of the endotracheal tube holder 100.

The body portion 110 can also include additional components that allow comfortable attachment of the endotracheal tube holder 100 to a patient. For example, as shown in FIGS. 2-4, the body portion 110 can include two widened distal portions 300 at opposite ends for placement against the patient's body, e.g., against the patient's cheeks or head. Moreover, in certain embodiments, at least one of the widened distal portions 300 may include an auxiliary tube holder 310. Alternatively, both widened distal portions 300 may include the auxiliary tube holder 310. In some embodiments, the auxiliary tube holder 310 is positioned away from the edges of the body portion 110 so that an auxiliary tube coming out from any edge of the body portion can be conveniently attached and secured. The auxiliary tube holder 310 may be a notch, in which the tube may be secured, and may be placed to orient an auxiliary tube away from the endotracheal tube along a longitudinal axis of the body portion 110 so that the auxiliary tube does not interfere with the securing and maintenance procedures of the endotracheal tube or add discomfort to the patient. The auxiliary tube holder 310 may be placed along a central axis of the widened distal portion 300 such that the position of the auxiliary tube holder with respect to the endotracheal tube holder is not relevant, offering versatility to the user to accommodate multiple positions. For example, the auxiliary tube may extend outward from the patient and from above or below the body portion 110.

The body portion 110 can further include a securing portion to attach the endotracheal tube holder 100 to a patient. For example, one of the distal portions can include a band-locking cleat 320 that allows for securing the body portion 110 to the patient. The band-locking cleat 320 includes a first opening 330 having a band-receiving region 331 that receives a band and a band-securing region 332 that friction-fits the band, and a second opening 340 that receives the band from the band-securing region 332 of the first opening 330. The band may be tightened or loosened around the patient by moving the band through the first opening 330. In certain embodiments, a channel 341 breaks the second opening 340 to an edge of the distal portion 300 so that the band can be moved into or removed out of the second opening 340 via the channel 341. The band may be secured by moving the band from the band-securing region 332 into the second opening 340 via the channel 341 and released by first removing the band away from the second opening 340 via the channel 341 and further moving the band from the band-securing region 332 to the band-receiving region 331. Both of the distal portions 300 may have the band-locking cleat 320, or alternatively, one distal portion may have the band-locking cleat 320 and the other distal portion may have the band permanently attached. The band can be a flexible elastic band, or can be made from a resilient fabric, strap, or cloth, such as polypropylene fiber cloth or a nylon woven braid. In some embodiments, the band may have a thickness of about 2 mm. The band allows a securing member 321 near the band-securing region 332 to dig into and locks the band from moving. The band-locking cleat 320 allows easy tightening, loosening, and securing of the band by using just one hand of a user or a medical practitioner, relieving the other hand for holding a device, such as the intubated endotracheal tube and/or an endotracheal tube holder. This easily operable band-locking cleat 320 thus allows time and labor efficient practice of securing a device to a patient, such as an endotracheal tube holder, especially desirable under emergency or “field” settings, where easy and quick securing mechanisms are crucial for life saving.

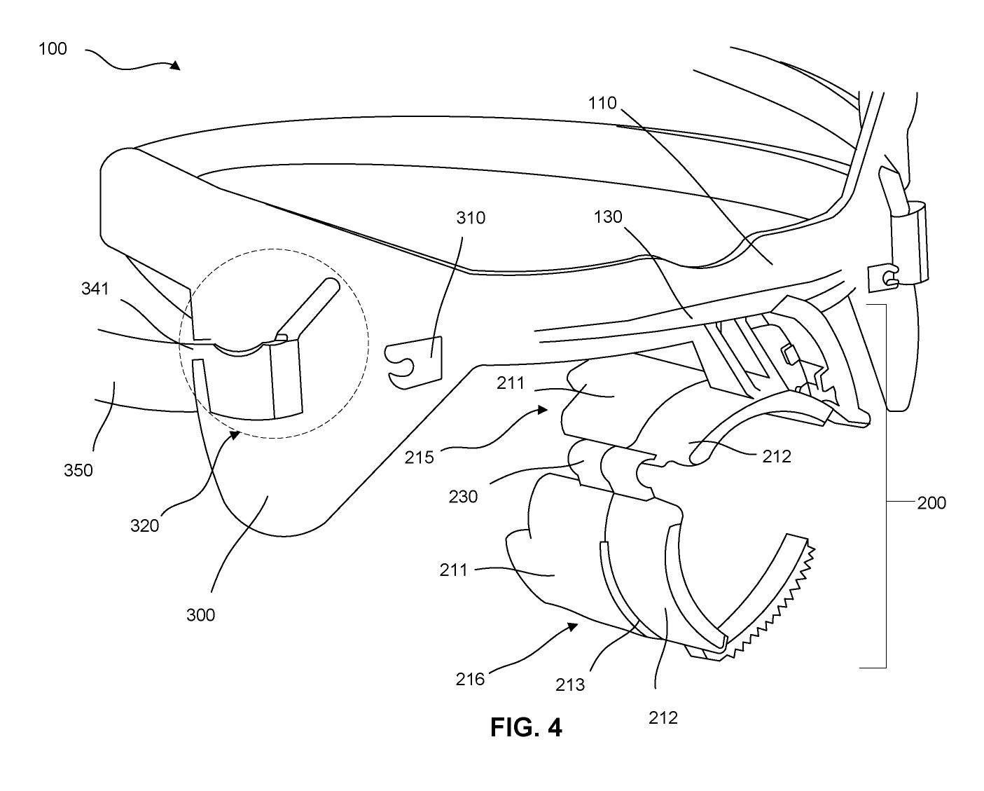

As shown in FIG. 5, the body portion 110 can also include pad regions 140 to further increase comfortable attachment of the endotracheal tube holder to a patient. For example, more than two pad regions 140 on a patient-facing side of the body portion 110 may be discretely distributed along the body portion. The pad regions 140 are preferably made of a soft material to provide a cushion against the harder surface of the body portion 110. The separation of the pad regions reduces pressure points on the patient while alleviating pressure caused by the body portion 110. The separation of the pad regions further allows access for suction, inspection, oral care, and maintaining cleanliness of the endotracheal tube and the integrity of tissues and surfaces involved. The discrete pad regions 140 can be of various sizes and shapes, e.g., circular, oval, or rectangular, and may also be detachable and adjustable by a user or a medical practitioner based on the needs of the specific medical situation. In addition, the pad regions may include an adhesive or tacky surface that prevents movement or sliding of the endotracheal tube holder. Alternatively, one continuous pad region 140 may be aligned along the longitudinal direction of the body portion 110 (not shown).

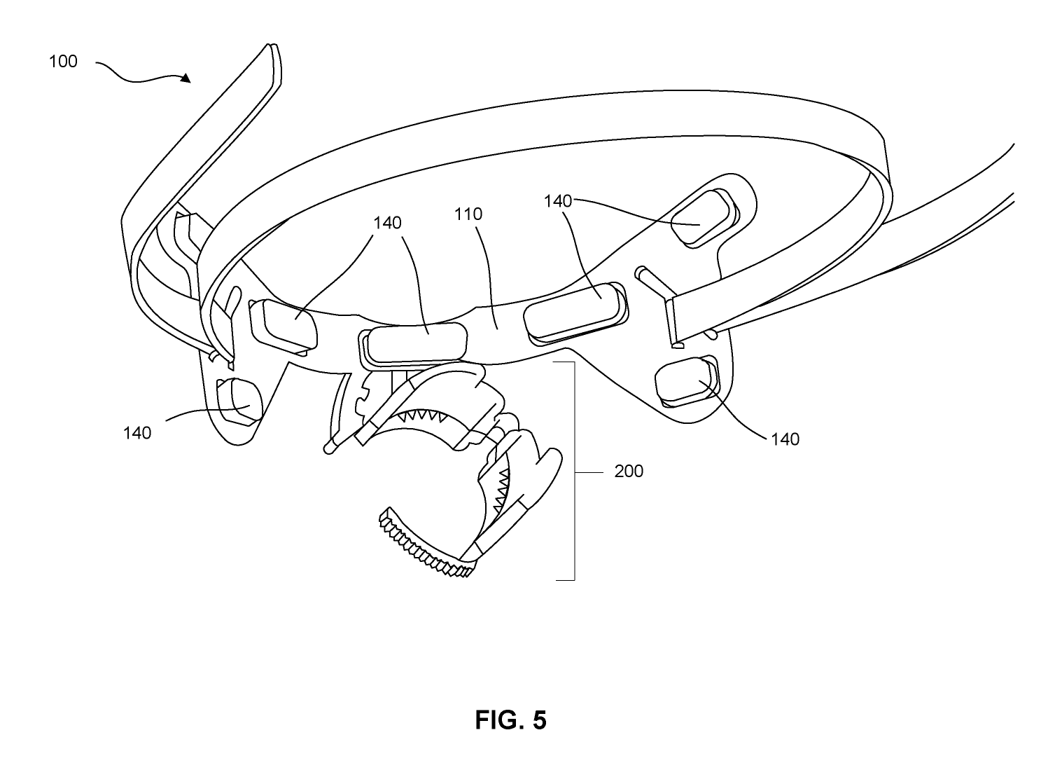

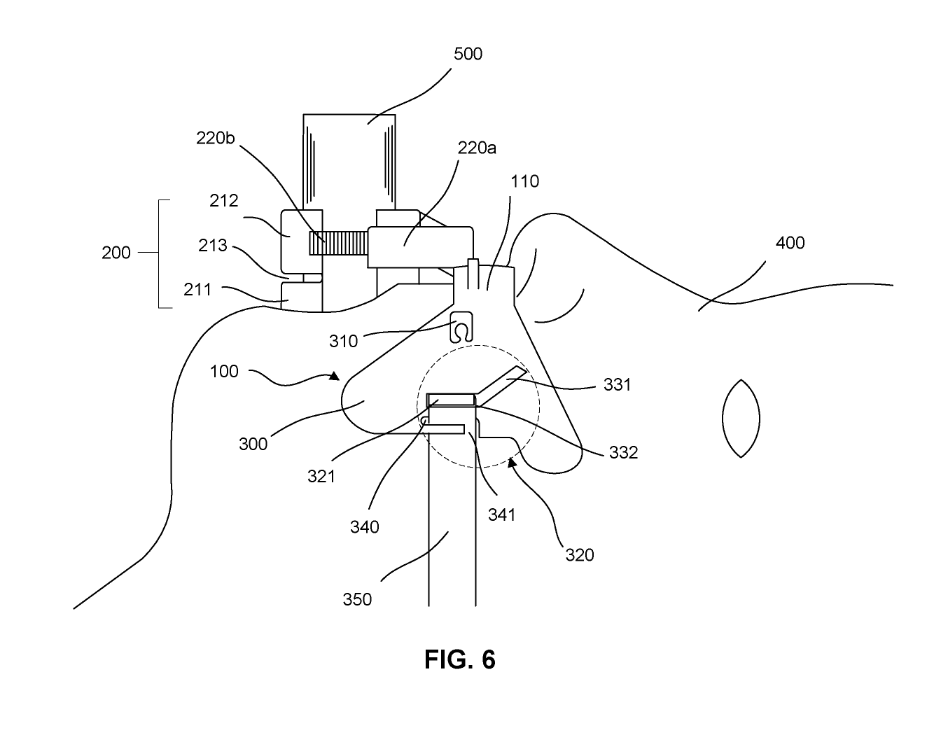

FIG. 6 provides a side view of an assembled endotracheal tube holder 100 holding an endotracheal tube 500 against the face of a patient 400, according to an exemplary embodiment consistent with the present disclosure. In FIG.6, a user or a medical practitioner has secured an intubated endotracheal tube 500 using the tube-grasping portion 200 of the endotracheal tube holder 100, and secured the endotracheal tube holder 100 to the face of the patient 400 by tightening and securing a band 350 through a band-locking cleat 320 of the endotracheal tube holder 100. In some embodiments, the band 350 is pre-threaded through the band-securing region 332 of the first opening of the band-locking cleat 320 before use. Thus, the user or the medical practitioner can pull the band 350 through the band-securing region 332 towards the earlobe of the patient to tighten the band 350 and secure the endotracheal tube holder 100 to the patient using one hand while using the other hand to hold the endotracheal tube holder 100 in place. After tightening the band 350 around the patient's neck or head, the user or the medical practitioner can further secure the band 350 and the endotracheal tube holder 100 by moving the band from the band-securing region 332 of the first opening to the second opening 340 via the channel 341. When the user or the medical practitioner decided to remove the endotracheal tube 500 and the endotracheal tube holder 100, the user or the medical practitioner may use one hand to hold the endotracheal tube holder 100 while using the other hand to release and loosen the band 350 by first moving the band 350 out of the second opening 340 via the channel 341, and then pulling the band away from the band-securing region 332 into the band-receiving region 331 of the first opening. During endotracheal intubation, an auxiliary tube may be used and secured by the auxiliary tube holder 310 on the distal portion 300 of the endotracheal tube holder 100. As shown in FIG. 6 and FIG. 7, the circumference of an endotracheal tube 500 is partially encompassed by the tube-grasping portion 200. The base member 220a adjustably latches with the movable member 220b of the locking assembly 220 via a plurality of ratcheting teeth 223 such that a desired degree of pressure and friction can be applied to a tube of any size to secure the tube.

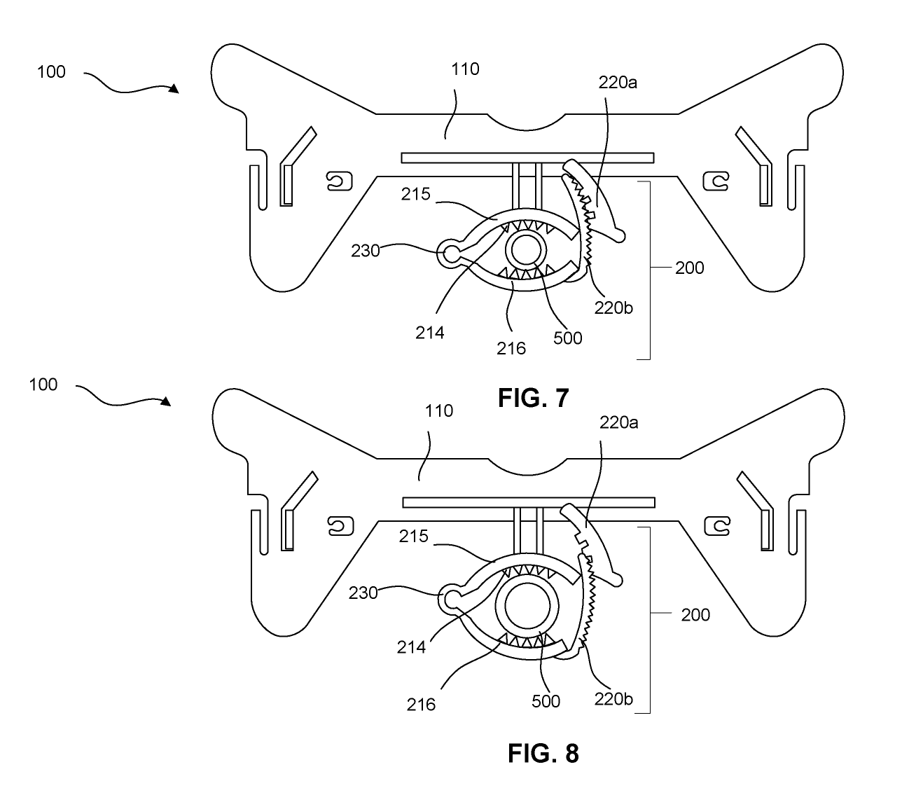

FIG. 7 and FIG. 8 show the endotracheal tube holder 100 securing a regular-sized endotracheal tube and a Supraglottic tube respectively according to two exemplary embodiments consistent with the present disclosure. The cooperation of the upper contact member 215, the lower contact member 216, and the movable member 220b that can be adjustably latched with the base member 220a enables the endotracheal tube holder 100 to secure the tube 500. The partial elliptic cylindrical shapes of the upper contact member 215 and the lower contact member 216 allow the endotracheal tube holder 100 to secure a tube of any size selected according to the specific medical situation by complementarily contacting and grabbing part of the outer circumference of the tube 500. The plurality of pointed grabbing teeth 214 on the interior sides of the upper contact member 215 and the lower contact member 216 may serve as contacting regions and increase pressure and friction between the tube clamp 210 and the tube.

Endotracheal tube holders consistent with the present disclosure prevent an endotracheal tube from being moved by a patient's bite and/or movement. Endotracheal tube holders consistent with the present disclosure are advantageously designed to adaptively accommodate and secure medical tubes of wide range of sizes, including the Supraglottic tubes, which minimize airway trauma while maximizing ventilation and allow passage into the esophagus and/or the stomach. Endotracheal tube holders consistent with the present disclosure may also have at least one auxiliary tube holder advantageously designed that conveniently secures an auxiliary tube coming out from any edge of the endotracheal tube holder and prevents the auxiliary tube from interfering with the securing and maintenance procedures of the endotracheal tube. Endotracheal tube holders consistent with the present disclosure may be made as a single piece, avoiding time-consuming assembly and/or disassembly of individual pieces in emergency medical situations. The band-locking cleat and/or the locking assembly consistent with the present disclosure advantageously enables one user or one medical practitioner to secure the endotracheal tube holder to the patient with only two hands, one for holding the endotracheal tube and/or the endotracheal tube holder while the other for tightening and securing.

Securing methods for providing a device to an object or a patient consistent with the present disclosure enable easily operable and time-efficient attachment and securing of the device. Securing methods consistent with the present disclosure provide a device preferably having at least one band-locking cleat that conveniently attaches and secures the device by allowing pulling and securing a strap or band. The band-locking cleat of the securing methods consistent with the present disclosure preferably has a first opening comprising a band-receiving region that receives the band and a band-securing region that friction-fits the band, and a second opening that receives the band from the band-securing region of the first opening. Securing methods consistent with the present disclosure thus allow the band to be moved or pulled with one hand through the first opening and be moved into or removed out of the second opening via a channel that is advantageously designed to break the second opening to an edge of the device. Moreover, securing methods consistent with the present disclosure preferably have the band pre-threaded through the first opening and placed within the band-securing region to be ready for a user or a medical practitioner to easily provide and secure the device with only two hands, one for holding the device while the other for tightening and securing.

It will be apparent to those skilled in the art that additional various modifications and variations can be made consistent with the present disclosure. Other embodiments will be apparent to those skilled in the art from consideration of the specification and practice of the disclosure. It is intended that the specification and examples be considered as exemplary only, with a true scope and spirit of the disclosure being indicated by the following claims.

What is claimed is:

1. An endotracheal tube holder comprising:
an elongated body portion; and
a tube-grasping portion comprising:
a tube clamp having a bite-block portion and a tube-securing portion and at least one channel separating the bite-block portion from the tube-securing portion, the tube-securing portion having a locking assembly, and at least one hinge.

2. The endotracheal tube holder of claim 1, further comprising at least one arm connecting the tube-grasping portion to the body portion.

3. The endotracheal tube holder of claim 2, wherein the body portion comprises a reinforcing rib extending along a longitudinal axis of the body portion and connected to the at least one arm.

4. The endotracheal tube holder of claim 3, wherein the reinforcing rib is further connected to a base member of the locking assembly.

5. The endotracheal tube holder of claim 4, wherein the reinforcing rib further includes a finger rest that provides leverage for closing the locking assembly.

6. The endotracheal tube holder of claim 3, wherein the tube clamp comprises an upper contact member and a lower contact member pivotably connected via the at least one hinge.

7. The endotracheal tube holder of claim 6, wherein a first hinge connects the upper contact member and the lower contact member of the tube-securing portion, and a second hinge connects the upper contact member and the lower contact member of the bite-block portion.

8. The endotracheal tube holder of claim 6, wherein the tube-securing portion comprises a plurality of teeth on the interior sides of the upper contact member and lower contact member to secure a tube therebetween.

9. The endotracheal tube holder of claim 6, wherein at least a part of the interior sides of the upper contact member and/or lower contact member have a frictional and/or adhesive surface to increase a gripping force to secure a tube therebetween.

10. The endotracheal tube holder of claim 6, wherein the shapes of the upper contact member and the lower contact member are partial elliptic cylindrical forms.

11. The endotracheal tube holder of claim 1,
wherein the locking assembly comprises a base member connected to the body portion and a movable member having a plurality of ratcheting teeth;
wherein the movable member is connected to the tube-securing portion; and
wherein the base member operatively latches to the movable member via the plurality of ratcheting teeth.

12. The endotracheal tube holder of claim 3, wherein the body portion has two widened distal portions at opposite ends for lying against the face of a patient.

13. The endotracheal tube holder of claim 12, wherein at least one of the widened distal portions comprises an auxiliary tube holder.

14. The endotracheal tube holder of claim 13, wherein both widened distal portions comprise the auxiliary tube holder.

15. The endotracheal tube holder of claim 13, wherein the auxiliary tube holder is positioned away from the edges of the body portion.

16. The endotracheal tube holder of claim 13, wherein the auxiliary tube holder is a notch.

17. The endotracheal tube holder of claim 13, wherein the auxiliary tube holder orients an auxiliary tube along the longitudinal axis of the body portion.

18. The endotracheal tube holder of claim 12, wherein at least one of the distal portions comprises a band-locking cleat that secures the endotracheal tube holder to the face of a patient, the band-locking cleat comprising:
a first opening having a band-receiving region that receives a band and a band-securing region that friction-fits the band; and
a second opening that receives the band from the band-securing region of the first opening;
wherein the band can be tightened or loosened around the patient by moving the band through the first opening; and
wherein a channel breaks the second opening to an edge of the distal portion so that the band can be moved into or removed out of the second opening via the channel.

19. The endotracheal tube holder of claim 18, wherein the band is secured by moving the band from the band-securing region into the second opening via the channel.

20. The endotracheal tube holder of claim 18, wherein the band is released by first removing the band away from the second opening via the channel and then moving the band from the band-securing region to the band-receiving region.

21. The endotracheal tube holder of claim 18, wherein both of the distal portions have the band-locking cleat.

22. The endotracheal tube holder of claim 18, wherein one distal portion has the band-locking cleat and the other distal portion has the band permanently attached.

23. The endotracheal tube holder of claim 1, wherein the body portion further comprises more than two pad regions on a patient-facing side of the body portion.

24. The endotracheal tube holder of claim 23, wherein the pad regions are discretely distributed on the body portion.

25. An endotracheal tube holder comprising:
a tube-grasping portion; and
an elongated body portion comprising two widened distal portions for lying against the face of a patient;
wherein at least one of the distal portions comprises a band-locking cleat that secures the endotracheal tube holder to the face of a patient, the band-locking cleat comprising:
a first opening having a band-receiving region that receives a band, and a band-securing region that friction-fits the band; and
a second opening that receives the band from the band-securing region of the first opening;
wherein the band can be tightened or loosened around the patient by moving the band through the first opening; and
wherein a channel breaks the second opening to an edge of the distal portion so that the band can be moved into or removed out of the second opening via the channel.

26. The endotracheal tube holder of claim 25, wherein the band is secured by moving the band from the band-securing region into the second opening via the channel.

27. The endotracheal tube holder of claim 25, wherein the band is released by first removing the band away from the second opening via the channel and then moving the band from the band-securing region to the band-receiving region.

28. The endotracheal tube holder of claim 25, wherein both of the distal portions have the band-locking cleat.

29. The endotracheal tube holder of claim 25, wherein one distal portion has the band-locking cleat and the other distal portion has the band permanently attached.

30. A band-locking cleat for securing a device to an object or a person comprising:
a first opening having a band-receiving region that receives a band and a band-securing region that friction-fits the band; and
a second opening that receives the band from the band-securing region of the first opening;
wherein the band can move through the first opening; and
wherein a channel breaks the second opening to an edge of the device so that the band can be moved into or removed out of the second opening via the channel.

31. The band-locking cleat of claim 30, wherein the band is secured by moving the band from the band-securing region into the second opening via the channel.

32. The band-locking cleat of claim 30, wherein the band is released by first removing the band away from the second opening via the channel and then moving the band from the band-securing region to the band-receiving region of the first opening.

33. A method for providing a device having at least one band-locking cleat, the band-locking cleat having:
a first opening comprising a band-receiving region that receives a band, and a band-securing region that friction-fits the band; and
a second opening that receives the band from the band-securing region of the first opening;
wherein the band can move through the first opening;
wherein a channel breaks the second opening to an edge of the device so that the band can be moved into or removed out of the second opening via the channel; and
wherein the band is pre-threaded through the first opening and placed within the band-securing region.
